(19) United States
(12) Patent Application Publication
(10) Pub. No.: US 2010/0256152 A1
(43) Pub. Date: Oct. 7, 2010

(54) NOVEL PHARMACEUTICAL COMPOSITION FOR TREATMENT OF SCHIZOPHRENIA

(75) Inventor: Shinji TAKAHASHI, Tokyo (JP)

Correspondence Address: SUGHRUE-265550, 2100 PENNSYLVANIA AVE. NW, WASHINGTON, DC, 20037-3213, US

(73) Assignee: ASTELLAS PHARMA INC., Tokyo (JP)

(21) Appl. No.: 12/753,422

(22) Filed: Apr. 2, 2010

Related U.S. Application Data
(60) Provisional application No. 61/275,695, filed on Apr. 3, 2009.

(51) Int. Cl. A61K 31/53; C07D 401/12; A61P 25/18

(52) U.S. Cl. 514/245; 544/198

(57) ABSTRACT

The present invention is useful for providing an excellent pharmaceutical composition for prevention and/or treatment of schizophrenia, containing a BEC1 potassium channel inhibitor or a pharmaceutically acceptable salt thereof as an active ingredient, and is particularly useful for providing a pharmaceutical composition for prevention and/or treatment of the positive symptoms, negative symptoms, cognitive impairments and the like of schizophrenia.

### TECHNICAL FIELD

The present invention relates to a novel pharmaceutical use of BEC1 potassium channel inhibitor as an agent for treating schizophrenia.

### BACKGROUND ART

Schizophrenia is one of major mental disorders, is a disease with poor prognosis, and has a relatively high lifetime prevalence, as high as 0.7 to 2.0% (PLoS Med. 2:413-433, 2005, herein incorporated by reference). The symptoms of schizophrenia are classified into positive symptoms, negative symptoms, cognitive impairments and mood disorder. The treatment of schizophrenia utilizes psychotherapy, occupational therapy and pharmacotherapy. Among these, pharmacotherapy achieves an important role. However, schizophrenic patients still suffer from the problems of the disease becoming recurrent, chronic and incurable, or of tardive dyskinesia or extrapyramidal adverse side effects of antipsychotics.

In the pharmacotherapy for schizophrenia, antipsychotics are primarily used. The antipsychotics may be classified into first generation antipsychotics and second generation antipsychotics. The first generation antipsychotics are central dopamine receptor antagonists, and particularly dopamine D2 receptor antagonists. Specifically, chlorpromazine, haloperidol, bromperidol, perphenazine and the like may be mentioned. On the other hand, the second generation antipsychotics include those having additional blocking action against serotonin receptors in addition to that against dopamine D2 receptors (risperidone, perospirone, ziprasidone, and the like), or those having additional blocking action against many other receptors (clozapine, olanzapine, and the like), those acting as partial agonists for dopamine D2 receptors (aripiprazole and the like), and the like. For any of those antipsychotics, the improving effects are considered to be still insufficient, and emergence of adverse side effects based on the dopamine receptor blocking action has become a problem (Japanese Journal of Clinical Psychopharmacology, 11:1089-1011, 2008, herein incorporated by reference).

Potassium channels are proteins which are present in the plasma membrane of cells and selectively pass potassium ions, and are conceived to be in charge of an important role for the control of membrane potential in cells. In particular, the potassium channels contribute to the neurotransmission of central and peripheral nerves, heart pace-making, contraction of muscles, and the like, by regulating the frequency, durability and the like of the action potential in neurons and muscle cells.

When the channels are classified based on the opening-closing mechanism, voltage-dependent potassium channels, inwardly rectifying potassium channels, calcium-dependent potassium channels, receptor coupled potassium channels, and the like have been identified hitherto. Among these, the voltage-dependent potassium channels have a characteristic of being opened when the membrane potential is depolarized. Typically, potassium ions exist in a non-equilibrium state of about 5 mM in the extracellular moiety and about 150 mM in the intracellular moiety. For this reason, when the voltage-dependent potassium channels open due to depolarization, potassium ions are discharged from the intracellular part to the extracellular part, and consequently induce recovery (repolarization) of the membrane potential. Therefore, a decrease in the excitability of neurons and muscle cells is induced, concomitantly with the opening of the voltage-dependent channels (Ionic Channels of Excited Membranes, Sinauer Associates, Sunderland, 1992, herein incorporated by reference).

A compound modifying the opening of the voltage-dependent channels regulates various physiological phenomena by regulating the excitability of neurons, muscle cells and the like, and also has a possibility of serving as a therapeutic drug for various diseases. For example, 4-aminopyridine which is an inhibitor of A-type voltage-dependent potassium channels found in nerve cells, is known to induce epilepsy by increasing the nerve excitability (Epilepsy Res. 11:9-16, 2002, herein incorporated by reference). Furthermore, dofetilide which is an inhibitor of hERG potassium channels expressed in the heart among the voltage-dependent potassium channels, is used as a drug for treatment arrhythmia based on the controlling of the excitability of myocardial cells (J. Pharmacol. Exp. Ther. 256:318-324, 1991, herein incorporated by reference).

The potassium channel as set forth in SEQ ID NO:2 in Example 1 of U.S. Pat. No. 6,326,168 (hereinafter, indicated as BEC1 or BEC1 potassium channel) is a voltage-dependent potassium channel showing a distribution of expression localized in the brain (U.S. Pat. No. 6,326,168 is herein incorporated by reference). Expression of this channel is conspicuous in the hippocampus or the cerebral cortex. The hippocampus and cerebral cortex are regions suggested to be strongly associated with learning and memory (The Neuron: Cell and Molecular Biology, Oxford University Press, New York, N.Y., 1991, herein incorporated by reference).

From this, there is conceived a possibility that the BEC1 potassium channel is associated with learning and memory. In fact, it was revealed with regard to a transgenic mouse having the BEC1 channel described in U.S. Pat. No. 7,375,222 highly expressed in the hippocampus and the cerebral cortex, that the mouse has a decreased learning ability in the Morris water maze learning test and the passive avoidance learning test (U.S. Pat. No. 7,375,222 is herein incorporated by reference). From this fact, it is conceived that an inhibitor of BEC1 potassium channel enhances learning and memory, and thus is considered to be highly promising as a therapeutic drug for dementia.

A number of potassium channel inhibitors have been reported hitherto, but the compounds reported to inhibit BEC1 potassium channel are only the 2,4,6-triamino-1,3,5-triazine derivatives described in U.S. Pat. No. 7,375,222, herein incorporated by reference. Furthermore, it is disclosed in WO 2002/050066 that certain types of 1,3,5-triazine-2,4,6-triamine derivatives have protein kinase inhibitory activity and are useful as agents for treating Alzheimer's disease or Parkinson's disease (WO 2002/050066 is herein incorporated by reference). However, there is no report to date on a finding suggesting that the BEC1 channel inhibitors show usefulness for diseases other than dementia, for example, schizophrenia.

### SUMMARY OF THE INVENTION

An object of the present invention is to provide a therapeutic agent for schizophrenia having a novel mechanism of action which is different from conventional antipsychotics.

In order to achieve the above-mentioned object, the inventors of the present invention conducted research based on a unique idea, and found that BEC1 potassium channel inhibitors exhibit a remarkable therapeutic effect on schizophrenia, thus completing the present invention.

According to an aspect of the present invention, there is provided a pharmaceutical composition for prevention and/or treatment of schizophrenia, containing an effective amount of a BEC1 potassium channel inhibitor or a pharmaceutically acceptable salt thereof, and a pharmaceutically acceptable carrier.

According to another aspect of the present invention, there is provided a prophylactic agent and/or therapeutic agent for schizophrenia, containing a BEC1 potassium channel inhibitor or a pharmaceutically acceptable salt thereof as an active ingredient.

According to another aspect of the present invention, there is provided a BEC1 potassium channel inhibitor or a pharmaceutically acceptable salt thereof for the prevention and/or treatment of schizophrenia.

According to still another aspect of the present invention, there is provided a use of a BEC1 potassium channel inhibitor or a pharmaceutically acceptable salt thereof for the manufacture of a medicament for treating schizophrenia.

According to still another aspect of the present invention, there is provided a method of preventing and/or treating schizophrenia, comprising administering an effective amount of a BEC1 potassium channel inhibitor or a pharmaceutically acceptable salt thereof in a patient in need of such disease.

According to still another aspect of the present invention, there is provided a method for preparing a pharmaceutical composition for treating schizophrenia, the method comprising mixing a BEC1 potassium channel inhibitor or a pharmaceutically acceptable salt thereof, and a pharmaceutically acceptable excipient.

According to still another aspect of the present invention, there is provided a commercial package comprising a pharmaceutical composition comprising a BEC1 potassium channel inhibitor or a pharmaceutically acceptable salt thereof as an active ingredient, and an instruction describing that the BEC1 potassium channel inhibitor or a pharmaceutically acceptable salt thereof can be used or should be used to treat schizophrenia.

### DETAILED DESCRIPTION OF THE PRESENT INVENTION

Preferred embodiments of the present invention will be presented in the following.

(1) A pharmaceutical composition for prevention and/or treatment of schizophrenia, containing an effective amount of a compound of formula (I):

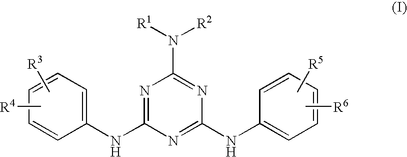

wherein the symbols are as follows.

R¹ and R², which may be the same or different, each represents H, OH, lower alkyl-O—, aryl-CO—, NH₂, lower alkyl-NH which may be substituted with OH, (lower alkyl)₂N, a lower alkyl which may be substituted, or a heterocyclic group which may be substituted; and

R³, R⁴, R⁵ and R⁶, which may be the same or different, each represents (i) H, (ii) CN, (iii) NO₂, (iv) halogen, (v) lower alkyl which may be substituted with (1) CN, (2) halogen, or (3) OH, (vi) cycloalkyl, (vii) aryl which may be substituted with lower alkyl, (viii) a heterocyclic group which may be substituted with lower alkyl, (ix) R⁷R⁸N-(wherein R⁷ and R⁸ may be the same or different, and each represents (1) H, (2) aryl, or (3) lower alkyl which may be substituted with R⁹—O—CO— (wherein R⁹ represents (1) H, or (2) lower alkyl which may be substituted with aryl)), (x) (wherein R¹⁰ represents (1) H, (2) lower alkyl which may be substituted with aryl, HO—C₁₋₁₀ alkylene-O— or OH, or (3) aryl; and T¹ represents O or S), or (xi) R¹¹-T²- (wherein R¹¹ represents (1) OH, (2) R⁷R⁸N—, (3) lower alkyl-O—, (4) lower alkyl, (5) aryl, or (6) a heterocyclic group; and T² represents CO or SO₂),

or a pharmaceutically acceptable salt thereof, and a pharmaceutically acceptable carrier.

(2) The pharmaceutical composition according to (1), wherein R¹ and R², which may be the same or different, each represents H, or lower alkyl which may be substituted with a heterocyclic group which may be substituted; and R³, R⁴, R⁵ and R⁶, which may be the same or different, each represents (i) H, (ii) halogen, or (iii) R¹⁰-T¹- (wherein R¹⁰ represents lower alkyl, and T¹ represents O).

(3) The pharmaceutical composition according to (1) or (2), wherein R¹ and R², which may be the same or different, each represents H, or lower alkyl which may be substituted with a heterocyclic group selected from pyrimidine and pyridine, which may be substituted with a substituent selected from the group consisting of halogen, lower alkyl and lower alkyl-O—.

(4) The pharmaceutical composition according to (1) to (3), wherein R¹ represents H; and R² represents lower alkyl substituted with a heterocyclic group selected from pyrimidine and pyridine, which may be substituted with a substituent selected from the group consisting of halogen, lower alkyl and lower alkyl-O—; R³ and R⁶ each represents H; and R⁴ and R⁵, which may be the same or different, each represents (i) H, (ii) halogen, or (iii) R¹⁰-T¹- (wherein R¹⁰ represents lower alkyl; and T¹ represents O).

(5) The pharmaceutical composition according to (1) to (4), wherein R¹ represents H; R² represents lower alkyl substituted with pyrimidine which may be substituted with a substituent selected from the group consisting of halogen, lower alkyl and lower alkyl-O—; R³ and R⁶ each represents H; and R⁴ and R⁵, which may be the same or different, each represents (i) H, (ii) halogen, or (iii) R¹⁰-T¹- (wherein R¹⁰ represents lower alkyl; and T¹ represents O).

(6) The pharmaceutical composition according to (1) to (4), wherein R¹ represents H; R² represents lower alkyl substituted with pyridine which may be substituted with a substituent selected from the group consisting of halogen, lower alkyl and lower alkyl-O—; R³ and R⁶ each represents H; and R⁴ and R⁵, which may be the same or different, each represents (i) H, (ii) halogen, or (iii) R¹⁰-T¹- (wherein R¹⁰ represents lower alkyl; and T¹ represents O).

(7) The pharmaceutical composition according to (1) to (6), wherein the schizophrenia is selected from the group consisting of positive symptoms associated with schizophrenia (hallucinations, delusions, xenopathic experiences, disorganized speech, highly disorganized or catatonic behavior, and the like), negative symptoms associated with schizophrenia (affective flattening, poverty of thinking, apathy, autism, anhedonia, and the like), cognitive impairments associated with schizophrenia, and mood disorder associated with schizophrenia (depression, anxiety, and the like).

As for specific compounds of the formula (I) included in the present invention, the following compounds may be mentioned.
N-(4-fluorophenyl)-N′-phenyl-N″-(pyrimidin-2-ylmethyl)-1,3,5-triazine-2,4,6-triamine,
N,N′-bis(4-fluorophenyl)-N″-(pyrimidin-2-ylmethyl)-1,3,5-triazine-2,4,6-triamine,
N-(4-fluorophenyl)-N′-(4-methoxyphenyl)-N″-(pyrimidin-2-ylmethyl)-1,3,5-triazine-2,4,6-tiramine,
N,N′-bis(4-fluorophenyl)-N″-(pyrimidin-4-ylmethyl)-1,3,5-triazine-2,4,6-triamine, or
N-(4-fluorophenyl)-N′-[(2-fluoro-4-pyridyl)methyl]-N″-phenyl -1,3,5-triazine-2,4,6-triamine.

In regard to the above or following descriptions of the present specification, appropriate examples of various definitions included in the scope of the present invention will be described in detail as follows.

The term “lower alkyl” means linear or branched alkyl having 1 to 6 carbon atoms (hereinafter, abbreviated to C1-6), and includes, for example, methyl, ethyl, n-propyl, isopropyl, n-butyl, isobutyl, sec-butyl, tert-butyl, n-pentyl, n-hexyl groups and the like. In another embodiment, the lower alkyl is C1-4 alkyl, and in still another embodiment, the lower alkyl is methyl, ethyl, propyl, isopropyl, butyl, isobutyl, tert-butyl, pentyl or hexyl.

The term “halogen” means F, Cl, Br, or I.

The term “C₁₋₁₀ alkylene” means linear or branched C₁₋₁₀ alkylene, and includes, for example, methylene, ethylene, trimethylene, tetramethylene, pentamethylene, hexamethylene, heptamethylene, octamethylene, nonamethylene, decamethylene, propylene, methylmethylene, ethylethylene, 1,2-dimethylethylene, 1,1,2,2-tetramethylethylene group, and the like.

The term “cycloalkyl” means a C₃₋₁₀ saturated hydrocarbon cyclic group, and may be bridged. The cycloalkyl includes, for example, cyclopropyl, cyclobutyl, cyclopentyl, cyclohexyl, cycloheptyl, cyclooctyl, adamantyl groups, and the like. In another embodiment, the cycloalkyl is C₃₋₈ cycloalkyl, and in still another embodiment, the cycloalkyl is C₃₋₆ cycloalkyl.

The term “aryl” means a C₆₋₁₄ monocyclic to tricyclic aromatic hydrocarbon cyclic group, and includes, for example, phenyl and naphthyl. In another embodiment, the aryl is phenyl.

The term “heterocyclic” group means a 3- to 15-membered, in another embodiment, 5- to 10-membered, monocyclic to tricyclic heterocyclic group containing 1 to 4 heteroatoms selected from oxygen, sulfur and nitrogen, and includes a saturated cyclic group, an aromatic cyclic group, and a partially hydrogenated cyclic group. The sulfur or nitrogen atom, both of which are ring atoms, may be oxidized to form oxide or dioxide. Specific examples include monocyclic heteroaryl such as pyridyl, pyrrolyl, pyrazinyl, pyrimidinyl, pyridazinyl, imidazolyl, triazolyl, triazinyl, tetrazolyl, thiazolyl, pyrazolyl, isothiazolyl, oxazolyl, isoxazolyl, thiadiazolyl, oxadiazolyl, thienyl, or furyl; bicyclic heteroaryl such as indolyl, isoindolyl, benzimidazolyl, indazolyl, quinolyl, isoquinolyl, quinazolinyl, quinoxalinyl, phthalazinyl, benzothiazolyl, benzisothiazolyl, benzothiadiazolyl, benzoxazolyl, benzisoxazolyl, benzofuranyl or benzothienyl; tricyclic heteroaryl such as carbazolyl, dibenzo[b,d]furanyl, or dibenzo[b,d]thienyl; non-aromatic monocyclic heterocyclic ring such as azetidinyl, pyrrolidinyl, piperidyl, piperazinyl, azepanyl, diazepanyl, morpholinyl, thiomorpholinyl, tetrahydropyridinyl, oxetanyl, tetrahydrofuranyl, tetrahydropyranyl, dioxolanyl, dioxanyl, or tetrahydrothiopyranyl; non-aromatic bicyclic heterocyclic ring such as indolinyl, tetrahydroquinolyl, tetrahydroisoquinolyl, dihydrobenzimidazolyl, tetrahydrobenzimidazolyl, tetrahydroquinoxalinyl, dihydroquinoxalinyl, dihydrobenzoxazolyl, dihydrobenzoxazinyl, dihydrobenzofuryl, chromanyl, chromenyl, methylenedioxyphenyl, or ethylenedioxyphenyl; bridged heterocyclic rings such as quinuclidinyl; and the like. In another embodiment, the heterocyclic group is a 5- to 10-membered monocyclic or bicyclic heterocyclic group, and in still another embodiment, the heterocyclic group is a 5- to 6-membered monocyclic heterocyclic group, and in still another embodiment, the heterocyclic group is 5- to 6-membered monocyclic heteroaryl.

The “lower alkyl which may be substituted” and “heterocyclic group which may be substituted” mean that the “lower alkyl” and “heterocyclic group” may be respectively substituted with substituents including one or two or more groups shown below.

—OH, —NH₂, —NH(lower alkyl), —N(lower alkyl)₂, —CN, —COOH, NO₂, lower alkyl, —O-lower alkyl, halogen, cycloalkyl, aryl, and a heterocyclic group (wherein the aforementioned cycloalkyl, aryl and heterocyclic group may be substituted with one or two or more substituents selected from the following groups.

—OH, —NH₂, —NH(lower alkyl), —N(lower alkyl)₂, —CN, —COOH, NO₂, lower alkyl, —O-lower alkyl, halogen, cycloalkyl, aryl and a heterocyclic group).

The term “BEC1” or “BEC1 potassium channel” means a protein as set forth in SEQ ID NO:2, which has been known in U.S. Pat. No. 6,326,168 or U.S. Pat. No. 7,375,222.

The term “BEC1 potassium channel inhibitor” means a substance inhibiting the BEC1 potassium channel, and for example, it means a substance having an IC₅₀ value of 10 μM or less; in another embodiment, 1 μM or less; and in still another embodiment, 0.5 μM or less, based on the evaluation method described in Example 1. The BEC1 potassium channel inhibitor is obtained by subjecting a test compound to a representative screening method, for example, the method described in U.S. Pat. No. 6,326,168 or U.S. Pat. No. 7,375,222, herein incorporated by reference.

The compound of the formula (I) may have tautomers or geometric isomers, depending on the type of substituent. In the present specification, the compound of the formula (I) may be described only as one form of isomers in some cases, but the present invention also includes the other isomers, as well as separated isomers or mixtures thereof.

The compound of the formula (I) may also have asymmetric carbon atoms or axial asymmetry, and optical isomers based thereon may also exist. The present invention includes separated optical isomers of the compound of the formula (I), or mixtures thereof.

Furthermore, the present invention also includes pharmaceutically acceptable prodrugs of the compound represented by the formula (I). A pharmaceutically acceptable prodrug is a compound having a group which can be converted to the amino group, hydroxyl group, carboxyl group or the like (of the present invention) by solvolysis or under physiological conditions. Examples of the group forming a prodrug include the groups described in Prog. Med., 5, 2157-2161 (1985) or “Development of Pharmaceutical Products” (Hirokawa-Shoten, Ltd., 1990), Vol. 7 Molecular Design, 163-198, both are herein incorporated by reference.

The compound of the formula (I) may also form a salt with an acid addition salt depending on the type of substituent, and such salt is included in the present invention so long as it is a pharmaceutically acceptable salt. Specific examples include acid addition salts with an inorganic acid such as hydrochloric acid, hydrobromic acid, hydroiodic acid, sulfuric acid, nitric acid, or phosphoric acid; or an organic acid such as formic acid, acetic acid, propionic acid, oxalic acid, malonic acid, succinic acid, fumaric acid, maleic acid, lactic acid, malic acid, mandelic acid, tartaric acid, dibenzoyltartaric acid, ditoluoyltartaric acid, citric acid, methanesulfonic acid, ethanesulfonic acid, benzenesulfonic acid, p-toluenesulfonic acid, aspartic acid, or glutamic acid; and the like.

The compound of the formula (I) and/or pharmaceutically acceptable salts thereof can be obtained by the production method described in U.S. Pat. No. 7,375,222, herein incorporated by reference, or by a production method equivalent thereto.

A pharmaceutical composition containing the compound of the formula (I), or one or two or more of pharmaceutically acceptable salts thereof, as an active ingredient can be prepared by using pharmaceutical excipients, pharmaceutical carriers and the like that are conventionally used in the pertinent art, according to a conventionally used method.

Administration may be carried out by any of the oral administration mode by means of tablets, pills, capsules, granules, powders, liquids or the like, and the parenteral administration mode by means of injectable preparations via intraarticular, intravenous, intramuscular routes, suppositories, eye drops, eye ointments, transdermal liquids, ointments, transdermal adhesive patches, transmucosal liquids, transmucosal adhesive patches, inhalants or the like.

As solid compositions for oral administration, tablets, powders, granules and the like are used. In these solid compositions, one or two or more active ingredients are mixed with at least one inert excipient, for example, lactose, mannitol, glucose, hydroxypropylcellulose, microcrystalline cellulose, starch, polyvinylpyrrolidone, and/or magnesium metasilicate aluminate, and the like. The composition may also contain inert additives, for example, a gliding agent such as magnesium stearate, a disintegrant such as carboxymethyl starch sodium, a stabilizer, and a dissolution aid, according to standard methods. Tablets or pills may be coated, if necessary, with sugar coating or a film of a gastrosoluble or enterosoluble material.

Liquid compositions for oral administration include pharmaceutically acceptable emulsions, solutions, suspensions, syrups or elixirs, and the like, and include a generally used inert diluent, for example, purified water or ethanol. The liquid compositions may also contain, in addition to the inert diluent, an auxiliary agent such as a solubilizer, a wetting agent or a suspending agent, a sweetener, a flavor, an aromatic, or an antiseptic.

An injectable preparation for parenteral administration contains a sterile, aqueous or non-aqueous solution, suspension or emulsion. Examples of aqueous solvents include distilled water for injection and physiological saline. Examples of non-aqueous solvents include propylene glycol, polyethylene glycol, plant oils such as olive oil, alcohols such as ethanol, Polysorbate 80 (name in the Japanese Pharmacopoeia), and the like. These compositions may further include an isotonic agent, an antiseptic, a wetting agent, an emulsifier, a dispersant, a stabilizer, or a dissolution aid. These are sterilized by, for example, filtration through a bacteria-retaining filter, incorporation of a bactericide, or irradiation. Furthermore, these can be used such that a sterile solid composition is prepared, and then dissolved or suspended in sterilized water or in a sterile solvent for injection before use.

Topical preparations include ointments, plasters, creams, jellies, adhesive skin patches, sprays, lotions, eye drops, eye ointments and the like. The topical preparations contain generally used ointment bases, lotion bases, aqueous or non-aqueous liquids, suspensions, emulsions and the like. For example, as the ointment or lotion base, polyethylene glycol, propylene glycol, white petrolatum, bleached beeswax, polyoxyethylene hydrogenated castor oil, glycerin monostearate, stearyl alcohol, cetyl alcohol, lauromacrogol, sorbitan sesquioleate, and the like may be mentioned.

The transmucous preparations such as inhalants or transnasal preparations are used in a solid, liquid or semi-solid form, and can be produced according to conventionally known methods. For example, known excipients, and furthermore, a pH adjusting agent, an antiseptic, a surfactant, a gliding agent, a stabilizer or thickening agent, and the like may be appropriately added. Administration can be carried out by using appropriate devices for inhalation or insufflation. For example, the compound can be administered alone or as a powder of a prescribed mixture, or as a solution or suspension in combination with a pharmaceutically acceptable carrier, using a known device such as a metered dose inhaler, or a sprayer. A dry powder inhaler or the like may be for a single dose or multiple doses, and dry powders or powder-containing capsules can be used. Alternatively, the preparation may also be in the form of an appropriate ejector, for example, a pressurized aerosol spray using a suitable gas such as chlorofluoroalkane, hydrofluroalkane or carbon dioxide.

Typically, in the case of oral administration, the daily dosage is appropriately about 0.001 to 100 mg/kg, preferably 0.1 to 30 mg/kg, and more preferably 0.1 to 10 mg/kg, of body weight, and this is administered once, or in two to four divided portions. In the case of carrying out intravenous administration, the daily dosage is appropriately about 0.0001 to 10 mg/kg of body weight, and this is administered once or in many divided portions per day. As for the transmucous preparations, about 0.001 to 100 mg/kg of body weight is administered once or in many divided portions per day. The dosage is appropriately determined in accordance with the individuals, while taking symptoms, age, gender and the like into consideration.

The compound of the formula (I) can be used in combination with an agent for treating or preventing schizophrenia. This combination may be administered simultaneously or separately and sequentially, or even may be administered at a desired time interval. The preparation for simultaneous administration may be a blend preparation, or may be separately formulated.

### EXAMPLES

The following Reference Examples and Examples are intended to describe the present invention in more detail, and the present invention is not to be limited to the following Examples. Although the present invention is sufficiently described by the Reference Examples and Examples, those ordinarily skilled in the art will understand that various alterations or modifications are definitely possible. Therefore, as long as such alterations or modifications does not depart from the scope of the present invention, they are included in the present invention.

In the Reference Examples, Examples and tables described below, the following abbreviations will be used.

Ex: Example number, REx: Reference example number, No: Compound number, mp: Melting point, Data: Physicochemical data (FAB+: FAB-MS (M+H)⁺, EI: EI-MS (M)⁺, NMR-DMSOd₆: δ (ppm) of peaks from ¹H NMR in DMSO-d₆), DMF: N,N-dimethylformamide, DMSO: dimethylsulfoxide, THF: tetrahydrofuran, 4 M hydrogen chloride/dioxane solution: 4 mol/l hydrogen chloride dioxane solution, MeCN: acetonitrile, MeOH: methanol, EtOH: ethanol.

### Reference Example 1-1

75.0 g of chloroisocyanuric acid and 680 mL of THF were added to a 2-L flask, followed by addition of 51.10 g of potassium carbonate at −19° C. under stirring. 41.08 g of p-fluoroaniline that has been diluted with 75 mL of THF at −12.4° C. or lower, and 75 mL of THF were added thereto. The reaction was carried out at −12.8 to −14.4° C. for 1 hour, and 450 mL of water was added. Liquid separation was carried out at room temperature to separate the aqueous layer, 300 mL of water was added thereto, and liquid separation was carried out again to separate the aqueous layer. To the organic layer were added an aqueous solution obtained by adding 1) 600 mL of THF, and 2) 1.1 g of potassium carbonate in 308 mL of water, and liquid separation was carried out to separate the aqueous layer. To the organic layer was added 150 mL of water, liquid separation was carried out to separate the aqueous layer, and the organic layer was concentrated under reduced pressure until the remaining amount of the solution became 280 mL. To the concentrated solution was added 750 mL of MeCN, and the concentration operation was carried out three times under reduced pressure until the remaining amount of the solution became 280 mL. Subsequently, 600 mL of MeCN was added thereto under cooling, followed by addition of 34.43 g of aniline and 75 mL of MeCN at −5.9° C. or less, and addition of 47.79 g of N,N-diisopropylethylamine and 38 mL of MeCN at −9.2° C. Thereafter, the temperature was elevated to room temperature, and after stirring for 12 hours, 48.42 g of 2-aminomethylpyrimidine and 75 mL of MeCN were added thereto at room temperature, followed by addition of 57.35 g of N,N-diisopropylethylamine and 38 mL of MeCN at room temperature. The inner temperature was elevated to 82.4° C., followed by stirring for 4.5 hours, and 560 mL of water was added thereto at an inner temperature of 70° C. or higher, followed by cooling. The crystal precipitation at an inner temperature of 65.8° C. was confirmed, followed by stirring at room temperature overnight, and filtration. The obtained crystal was washed with a mixed solution of MeCN:water=2:1, and subsequently washed with 300 mL of water. The obtained crystal was dried at 50° C. for 1 day under reduced pressure to obtain 108.54 g of N-(4-fluorophenyl)-N′-phenyl-N″-(pyrimidin-2-ylmethyl)-1,3,5-triazine-2,4,6-triamine.

### Reference Example 1-2

414 L of methyl ethyl ketone and 23.00 kg of the N-(4-fluorophenyl)-N′-phenyl-N″-(pyrimidin-2-ylmethyl)-1,3,5-triazine-2,4,6-triamine were added to a reaction vessel 1, and dissolved at an inner temperature of 65.0° C. After filtration, the mixture was transferred to a reaction vessel 2, followed by heating again. 6.90 kg of fumaric acid and 115 L of EtOH were added to the reaction vessel 1, dissolved at an inner temperature of 58.3° C., transferred to the reaction vessel 2. After cooling, the crystallization was initiated at an inner temperature of 54.2° C., followed by stirring at 0° C. overnight. After filtration, the crystal was washed with 46 L of EtOH, and 30.34 kg of the obtained “crystal of the salt having a ratio of the N-(4-fluorophenyl)-N′-phenyl-N″-(pyrimidin-2-ylmethyl)-1,3,5-triazine-2,4,6-triamine to fumaric acid of 1:1” (type III crystal: wet) and 460 L of EtOH were added to the reaction vessel 2. They were stirred at an inner temperature of 52.4 to 69.2° C. in a suspension state for 42 hours, cooled, and stirred at room temperature overnight. After filtration, the obtained crystal was washed with 46 L of EtOH, and then dried at 60° C. for 4 days under reduced pressure to obtain 20.97 kg of a “crystal of an anhydrous salt having a ratio of the N-(4-fluorophenyl)-N′-phenyl-N″-(pyrimidin-2-ylmethyl)-1,3,5-triazine-2,4,6-triamine to fumaric acid of 2:1” (type I).

### Reference Example 2-1

To a mixed solution of 25 g of 2-pyrimidinecarbonitrile in 100 mL of acetic acid and 100 mL of ethyl acetate, 1 g of 10% palladium/carbon was added, and the mixture was stirred for 14 hours at room temperature in a hydrogen atmosphere at ambient pressure. The palladium/carbon was removed from the reaction mixture by filtration through Celite, and an operation of adding toluene to a residue obtained by distilling off the solvent, and concentrating the mixture, was repeated four times. MeCN was added to the obtained residue to solidify the residue, and the solids were collected by filtration, to obtain 15.7 g of 1-pyrimidin-2-ylmethylamine acetate as a colorless solid.

### 1-Pyrimidin-2-ylmethylamine acetate

NMR-DMSOd₆:

1.88 (3H, s), 3.91 (2H, brs), 4.1-5.3 (3H, m), 7.38 (1H, t, J=4.9 Hz), 8.78 (2H, d, J=4.9 Hz)

EI: 109

### Reference Example 2-2

To a solution of 4.71 g of 6-chloro-N,N′-bis(4-fluorophenyl)-1,3,5-triazine-2,4-diamine in 50 mL of MeCN, 2.507 g of 1-pyrimidin-2-ylmethylamine acetate and 5.2 mL of N,N-diisopropylethylamine were added, and the mixture was stirred for 17 hours at 75° C. The reaction mixture was cooled to room temperature, and then to the residue obtained by distilling off the solvent, ethyl acetate was added. The organic layer was washed with 5% aqueous citric acid solution and saturated brine, and dried over anhydrous magnesium sulfate, and then the solvent was distilled off. The obtained residue was purified by silica gel column chromatography (chloroform:MeOH=100:0 to 95:5), to obtain 6.0 g of a pale yellow amorphous material. This was dissolved in 180 mL of EtOH, 2 g of activated carbon was added, and the mixture was stirred for one hour. The activated carbon was removed by filtration through Celite, and the residue obtained by distilling off the solvent was solidified from 150 mL of aqueous EtOH (EtOH 80%), to obtain 4.84 g of N,N′-bis(4-fluorophenyl)-N″-(pyrimidin-2-ylmethyl)-1,3,5-triazine-2,4,6-triamine as a colorless solid.

1.5 g of N,N′-bis(4-fluorophenyl)-N″-(pyrimidin-2-ylmethyl)-1,3,5-triazine-2,4,6-triamine was dissolved in 300 mL of MeOH, and 2 mL of a 4 M hydrogen chloride/dioxane solution was added. Then, the solvent was distilled off, and the obtained residue was crystallized from ethanol, to obtain 1.66 g of a “salt of N,N′-bis(4-fluorophenyl)-N″-(pyrimidin-2-ylmethyl)-1,3,5-triazine-2,4,6-triamine and hydrogen chloride at a ratio of 1:2” as colorless crystals.

### Reference Example 2-3

To a solution of 1.0 g of N,N′-bis(4-fluorophenyl)-N″-(pyrimidin-2-ylmethyl)-1,3,5-triazine-2,4,6-triamine in 50 mL of ethanol was added a solution of 285.6 mg of fumaric acid in 5 mL of ethanol. Soon, the precipitation of a solid was initiated. This reaction solution was heated under reflux to completely dissolve the solid, followed by stirring while leaving it to be cooled. When the inner temperature was lowered to 60° C., an extremely small amount of the crystal of the salt having a ratio of N,N′-bis(4-fluorophenyl)-N″-(pyrimidin-2-ylmethyl)-1,3,5-triazine-2,4,6-triamine to fumaric acid of 1:1 was added thereto, followed by stirring at room temperature for 12 hours while leaving it to be cooled. The precipitated crystals were collected by filtration, washed with ethanol, and dried at 60° C. for 2 days under reduced pressure to obtain 970 mg of a “salt having a ratio of N,N′-bis(4-fluorophenyl)-N″-(pyrimidin-2-ylmethyl)-1,3,5-triazine-2,4,6-triamine to fumaric acid of 1:1” as colorless crystals.

The compounds of Reference Example 3 (“salt of N-(4-fluorophenyl)-N′-(4-methoxyphenyl)-N″-(pyrimidin-2-ylmethyl)-1,3,5-triazine-2,4,6-triamine and hydrogen chloride at a ratio of 1:2”) and Reference Example 4 (“composition of N,N′-bis(4-fluorophenyl)-N″-(pyrimidin-4-ylmethyl)-1,3,5-triazine-2,4,6-triamine.1.7 hydrogen chloride.0.2 diethyl ether.1.8H₂O”) as shown in the following Table 2 were synthesized in the same manner as in Reference Examples 2-1, 2-2 and 2-3.

The structures and property values of the Reference Example compounds are presented in the following Table 1 and 2.

TABLE 1

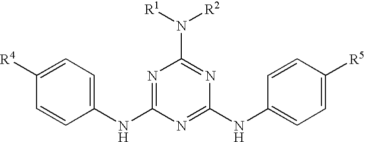

| REx | 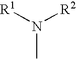 | R⁴ | R⁵ | Salt/Attached solvent | DATA |
|---|---|---|---|---|---|
| 1-1 | 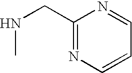 | F | H | — | NMR-DMSOd6 4.71-4.73 (2H, m), 6.91-7.26 (5H, m), 7.37 (1H, dd, J = 5.2 Hz, 4.8 Hz), 7.44-7.80 (5H, m), 8.78 (2H, d, J = 4.8 Hz), 9.01-9.05 (2H, m) FAB+: 389 Elemental Analysis. Calcd for C20H17FN8: C, 61.85; H, 4,41; N, 28.85; F, 4.89; Cl, 0.00. Found: C, 61.78; H, 4.43; N, 28.81; F, 4.95; Cl, 0.00 |
| 1-2 | 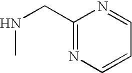 | F | H | 0.5 Fumaric acid | NMR-DMSOd6 4.71-4.73 (2H, m), 6.64 (1H, s), 6.91-7.23 (5H, m), 7.37 ppm (1H, dd, J = 5.2 Hz, 4.8 Hz), 7.44-7.80 (5H, m), 8.78 (2H, d, J = 4.8 Hz), 9.01-9.06 (2H, m), 13.06 (1H, br) FAB+: 389 Elemental Analysis. Calcd for C20H17FN8• 0.5C4H4O4: C, 59.19; H, 4, 29; N, 25.10; F, 4.26; 0, 7.17. Found: C, 59.09; H, 4.36; N, 25.19; F, 4.31. |
| 2-2 | 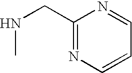 | F | F | 2HCl | NMR-DMSOd6 4.78 (2H, m), 7.10 (2H, brs), 7.25 (2H, t, J = 8.7 Hz), 7.3-7.8 (6H, m), 8.85 (2H, d, J = 4.9 Hz), 8.9-9.4 (1H, m), 10.39 (1H, s), 10.74 (1H, brs) Elemental Analysis. Calcd for C20H16F2N8• 2HC1: C, 50.12; H, 3.79; N, 23.33; F, 7.93; Cl, 14.79. Found: C, 50.06; H, 3.85; N, 23.38; F, 8.05; Cl, 14.91. mp: 183-186°C |

TABLE 2

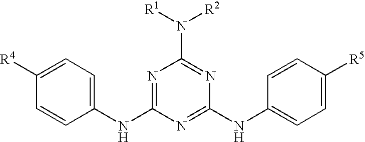

| REx | 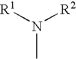 | R⁴ | R⁵ | Salt/Attached solvent | DATA |
|---|---|---|---|---|---|
| 2-3 | 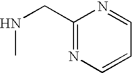 | F | F | 1 Fumaric acid | NMR-DMSOd6 4.70 (2H, d, J = 5.7 Hz), 6.64 (2H, s), 7.00 (2H, m), 7.10 (2H, t, J = 8.7 Hz), 7.38 (1H, t, J = 4.9 Hz), 7.57 (3H, brs), 7.80 (2H, brs), 8.80 (2H, d, J = 4.9 Hz), 9.0-9.2 (2H, m), 13.13 (2H, brs). Elemental Analysis. Calcd for C20H16F2N8C4H4O4: C, 55.17; H, 3.86; N, 21.45; F, 7.27. Found: C, 55.29; H, 4.05; N, 21.64; F, 7.32. |
| 3 | 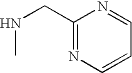 | F | OMe | 2HCl | NMR-DMSOd6 3.70-3.82 (3H, m), 4.6-5.0 (2H, m), 6.7-7.8 (10H, m), 8.85 (2H, d, J = 4.9 Hz), 9.0-9.7 (1H, m), 10.1-11.3 (2H, m) FAB+: 419 |
| 4 | 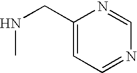 | F | F | 1.7HCl/ 0.2C₄H₁₀O/ 1.8H₂O | NMR-DMSOd6 4,65 (2H, brs), 6.8-7.3 (4H, m), 7.3-7.9 (6H, m), 8.6-9.0 (1H, m), 9.17 (1H, d, J = 1.2 Hz), 9.8-10.7 (2H, m) FAB+: 407 |

### Example 1

### Test Method

Method for measuring BEC1 inhibitory activity of compound utilizing 86Rb ion release amount as index

The channel activity of BEC1 was measured according to the method described in U.S. Pat. No. 6,326,168 or U.S. Pat. No. 7,375,222, herein incorporated by reference, utilizing the release of the ions of radioactive isotope 86Rb from BEC1 expressing cells as an index. Specifically, when BEC1 expressing cells which had taken in 86Rb ions were stimulated with 100 mM KCl, the radioactivity released from the same cells was designated as the channel activity of BEC1. 86Rb ions were incorporated into cells by culturing (3 hours, 37° C.) BEC1 stably expressing cells in the presence of 86RbCl (0.5 μCi/ml), and the unincorporated 86Rb ions were removed by washing the cells three times with HEPES buffered physiological saline (pH 7.4, 2.5 mM KCl). The same cells were incubated for 15 minutes at room temperature in the presence of a DMSO solution containing the test compound and HEPES buffered physiological saline, and then were further incubated for 5 minutes at room temperature in the presence of a 100 mM KCl-containing HEPES buffer solution (pH 7.4) containing the same compound. The extracellular fluid was recovered, and then the remaining cells were lysed in 0.1 N NaOH and recovered. The Cherenkov radioactivities of the extracellular fluid and the cell lysate were respectively measured, and the sum was designated as the total radioactivity. The release amount of 86Rb ions was expressed as the percentage of the radioactivity of the extracellular fluid with respect to the total radiation activity. The value obtained in the presence of the compound was designated as a test value, the value obtained in the absence of the compound was designated as a control value, and the value obtained when the cells were not stimulated with 100 mM KCl was designated as a blank value. The inhibitory action of the compound (as a free form which does not include salt) was indicated as the IC₅₀ value determined from the inhibition % (that is, (control value−test value)×100/(control value−blank value)). In addition, as for the BEC1 expressing cells, BEC1 stably expressing cells produced according to the method described in U.S. Pat. No. 6,326,168 or U.S. Pat. No. 7,375,222, herein incorporated by reference, using a dihydrofolate reductase (dhfr)-deficient strain of Chinese Hamster ovary cells, were used.

(Results)

The test results of representative compounds are presented in Table 3. The corresponding compounds were confirmed to have BEC1 potassium channel inhibitory action.

### Example 2

### Test Method

Verification of the therapeutic effect on schizophrenia was carried out using a methamphetamine induced hyperlocomotion model. Methamphetamine is a psychostimulant, and is known to cause symptoms that are similar to schizophrenia by increasing the transmission in the dopaminergic neurons. The abnormal behavior produced when methamphetamine is administered to an animal is generally used as a screening method for a therapeutic drug for schizophrenia (Oka et al., 1993, J. Pharmacol. Exp. Ther., 264:158-165, herein incorporated by reference). That is, a male ddY mouse was placed in an activity monitoring apparatus, and after 30 minutes, methamphetamine was administered. After administering methamphetamine, the mouse was immediately returned to the monitoring apparatus, and the activity for one hour from immediately after the return was measured. For the measurement of the activity, a Supermex sensor manufactured by Muromachi Kikai Co., Ltd. was used. A solvent (vehicle), or dilutions prepared by diluting the compounds described in Reference Examples 1-2 (test compound (1)), 2-2 (test compound (2a)), 3 (test compound (3)) and 4 (test compound (4)), and N-(4-fluorophenyl)-N′-[(2-fluoro-4-pyridyl) methyl]-N″-phenyl-1,3,5-triazine-2,4,6-triamine hydrochloride (test compound (5)), with a solvent at multiple concentrations (as a free form which does not include salt), were orally administered to the mice in each group. The solvent used was a 0.5% aqueous solution of methylcellulose. The statistical analysis was carried out between the solvent administered group and the drug administered groups, using Dunnett's test.

(Results)

The results of the methamphetamine induced hyperlocomotion suppressive action are presented in Table 3. The numerical values in the table represent the respective minimum effective doses for the compound administered groups (the smallest dose inducing a significantly small activity with respect to the activity of the solvent administered group). The test compounds (1) to (5) all suppressed methamphetamine induced hyperlocomotion. In other words, these five compounds were shown to have an effect of improving the symptoms of schizophrenia, especially the positive symptoms of schizophrenia.

### Example 3

### Test Method

Verification of the therapeutic effect on schizophrenia was carried out using a phencyclidine induced prolonged immobility model. Phencyclidine is a non-competitive antagonist of the N-methyl-D-aspartate receptor, a type of glutamate receptor, and abuse of this agent causes development of psychotic symptoms indistinguishable from those of schizophrenia. It was suggested that chronic treatment of phencyclidine enhances immobility in forced swimming test (Noda et al., 2000, Neuropsychopharmacol., 23:375-87, herein incorporated by reference). Forced swimming test is used as a screening assay for antidepressants, because immobility in this test is thought to reflect some aspect of depression such as lowering of motivation. Negative symptoms of schizophrenia also includes similar aspects, we could evaluate the efficacy of drugs for negative symptoms, such as apathy, using this test. Briefly, a male ddY mouse was gently placed in a cylindrical pool, and activity was measured for 3 min with SCANET (MV20-LAN, MELQUEST). Afterward immobility time was calculated with the SCANET file integrating software. Two days later, Mice were anesthetized with sodium pentobarbital. The Osmotic mini pumps (Alzet model 1002, DURECT Corporation) were subcutaneously implanted to mice. The pump infused saline or phencyclidine 1.2 mg/day/mouse with pumping rate of 0.25 μL/hour. Fourteen days after implantation, a test compound was administered to mice and an hour after administration, each mouse was gently placed in a cylindrical pool, and activity was measured for 3 min. Afterward immobility time was calculated. A solvent (vehicle), or dilutions prepared by diluting the test compounds (1) and (2b), with a solvent at multiple concentrations (as a free form which does not include salt), were orally administered to the mice in each group. The solvent used was a 0.5% aqueous solution of methylcellulose. The statistical analysis was carried out between the solvent administered group and the drug administered groups, using Dunnett's test.

(Results)

The results of the phencyclidine induced prolonged immobility improving action are presented in Table 3. The numerical values in the table represent the respective minimum effective doses for the compound administered groups (the smallest dose inducing a significantly short immobility time with respect to the immobility time of the solvent administered group). The test compounds (1) and (2b) suppressed phencyclidine induced prolongation of immobility time. In other words, these two compounds were shown to have an effect of improving the symptoms of schizophrenia, especially the negative symptoms of schizophrenia.

### Example 4

### Test Method

Verification of the therapeutic effect on schizophrenia was carried out using a water finding task in mice after neonatal treatment with phencyclidine. It was suggested that treatment with phencyclidine at the neonatal stage produced schizophrenic cognitive impairment (Depoortere et al., 2005, Neuropsychopharmacology, 30:1963-85, herein incorporated by reference). In addition, it has been reported that chronic treatment of PCP prolonged finding latency in the water-finding task (Mouri et al., 2007, Mol. Pharmacol., 180:152-60, herein incorporated by reference). Finding latency in the water-finding task provides a measure of latent learning in animals, which is thought to reflect attention. Attention is one of the most important domain of cognitive impairments in schizophrenia, we could evaluate the efficacy of drugs for cognitive impairments in schizophrenia using this task. Briefly, male ddY mice subcutaneously received PCP at a dose of 15 mg/kg or saline on postnatal day 7, 9 and 11. During the adult 7 weeks old, test compounds were orally administered to animals and 60 minutes later, the animals were placed in one corner of the open field and allowed to freely explore the training apparatus for 3 min. Immediately after the training trial, the animals were returned to their home cage and deprived of water until the test trial the next day. In the test trial, the mice were again placed individually in the test apparatus. The time between entering the alcove and drinking the water was measured. A solvent (vehicle), or dilutions prepared by diluting the test compounds (1) and (2b), with a solvent at multiple concentrations (as a free form which does not include salt), were orally administered to the mice in each group. The solvent used was a 0.5% aqueous solution of methylcellulose. The statistical analysis was carried out between the solvent administered group and the drug administered groups, using Dunnett's test.

(Results)

The results of the phencyclidine induced prolonged finding latency improving action are presented in Table 3. The numerical values in the table represent the respective minimum effective doses for the compounds administered groups (the smallest dose inducing a significantly short finding latency with respect to the finding latency of the solvent administered group). The test compounds (1) and (2b) suppressed phencyclidine induced prolongation of finding latency. In other words, these two compounds were shown to have an effect of improving the symptoms of schizophrenia, especially the cognitive impairment of schizophrenia.

### Test Compounds

Compound (1) means the compound of REx 1-2,
compound (2a) means the compound of REx 2-2,
compound (2b) means the compound of REx 2-3,
compound (3) means the compound of REx 3,
compound (4) means the compound of REx 4,
compound (5) means
N-(4-fluorophenyl)-N′-[(2-fluoro-4-pyridyl)methyl]-N″-phenyl-1,3,5-triazine-2,4,6-triamine hydrochloride, compound (6) means
N,N′-bis(4-fluorophenyl)-N″-[(2-fluoro-4-pyridyl)methyl]-1,3,5-triazine-2,4,6-triamine dihydrochloride, compound (7) means
N-(4-fluorophenyl)-N′-[(2-fluoro-4-pyridyl)methyl]-N″-(4-methylphenyl)-1,3,5-triazine-2,4,6-triamine hydrochloride, compound (8) means
N-(4-fluorophenyl)-N′-[(2-fluoro-4-pyridyl)methyl]-N″-(4-methoxyphenyl)-1,3,5-triazine-2,4,6-triamine hydrochloride, compound (9) means
N-(4-chlorophenyl)-N′-[(2-fluoro-4-pyridyl)methyl]-N″-(4-fluorophenyl)-1,3,5-triazine-2,4,6-triamine hydrochloride, compound (10) means
N-(4-fluorophenyl)-N′-[(2-fluoro-4-pyridyl)methyl]-N″-(3-methoxyphenyl)-1,3,5-triazine-2,4,6-triamine hydrochloride.
   The compounds (5)-(10) are described in U.S. Pat. No. 7,375,222.

TABLE 3

| | | Minimum effective dose | | |
|---|---|---|---|---|
| Test | IC₅₀ (μM) | (mg/kg p.o.) | | |
| Compound | (Example 1) | (Example 2) | (Example 3) | (Example 4) |
| 1 | 0.077 | 0.1 | 0.03 | 0.03 |
| 2a | 0.065 | 0.03 | — | — |
| 2b | — | — | 0.01 | 0.01 |
| 3 | 0.092 | 0.03 | — | — |
| 4 | 0.058 | 0.01 | — | — |
| 5 | 0.085 | 1.0 | — | — |
| 6 | 0.10 | — | — | — |
| 7 | 0.24 | — | — | — |
| 8 | 0.17 | — | — | — |
| 9 | 0.65 | — | — | — |
| 10 | 0.25 | — | — | — |

The data shows that BEC1 potassium channel inhibitors are useful for the prevention and/or treatment of schizophrenia. The pharmaceutical composition of the present invention is useful for providing an excellent prophylactic agent and/or therapeutic agent for schizophrenia, and is particularly useful for providing a prophylactic agent and/or therapeutic agent for the positive symptoms, negative symptoms and cognitive impairments and the like of schizophrenia.

While the invention has been described in detail and with reference to specific embodiments thereof, it will be apparent to one skilled in the art that various changes and modifications can be made therein without departing from the scope thereof.

1. A pharmaceutical composition for prevention and/or treatment of schizophrenia, comprising an effective amount of a BEC1 potassium channel inhibitor or a pharmaceutically acceptable salt thereof, and a pharmaceutically acceptable carrier.

2. The pharmaceutical composition according to claim 1, wherein the BEC1 potassium channel inhibitor is a compound of the following formula (I) or a pharmaceutically acceptable salt thereof: 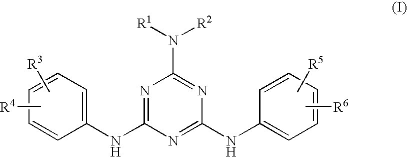
wherein the symbols are as follows;
R¹ and R², which may be the same or different, each represents H, OH, lower alkyl-O—, aryl-CO—, NH₂, lower alkyl-NH which may be substituted with OH, (lower alkyl)₂N, a lower alkyl which may be substituted, or a heterocyclic group which may be substituted; and
R³, R⁴, R⁵ and R⁶, which may be the same or different, each represents (i) H, (ii) CN, (iii) NO₂, (iv) halogen, (v) lower alkyl which may be substituted with (1) CN, (2) halogen, or (3) OH, (vi) cycloalkyl, (vii) aryl which may be substituted with lower alkyl, (viii) a heterocyclic group which may be substituted with lower alkyl, (ix) R⁷R⁸N-(wherein R⁷ and R⁸ may be the same or different, and each represents (1) H, (2) lower alkyl which may be substituted with aryl, or (3) R⁹—O—CO— (wherein R⁹ represents (1) H, or (2) lower alkyl which may be substituted with aryl)), (x) R¹⁰-T¹- (wherein R¹⁰ represents (1) H, (2) lower alkyl which may be substituted with aryl, HO—C₁₋₁₀ alkylene-O— or OH, or (3) aryl; and T¹ represents O or S), or (xi) R¹¹-T²- (wherein R¹¹ represents (1) OH, (2) R⁷R⁸N—, (3) lower alkyl-O—, (4) lower alkyl, (5) aryl, or (6) a heterocyclic group; and T² represents CO or SO₂).

3. The pharmaceutical composition according to claim 2, wherein the formula (I) is a compound wherein
R¹ and R², which may be the same or different, each represents H or lower alkyl which may be substituted; and
R³, R⁴, R⁵ and R⁶, which may be the same or different, each represents (i) H, (ii) halogen, or (iii) R¹⁰-T¹- (wherein R¹⁰ represents lower alkyl; and T¹ represents O),
or a pharmaceutically acceptable salt thereof.

4. A BEC1 potassium channel inhibitor or a pharmaceutically acceptable salt thereof for the prevention and/or treatment of schizophrenia.

5. The BEC1 potassium channel inhibitor according to claim 4, wherein the BEC1 potassium channel inhibitor is a compound of the following formula (I) or a pharmaceutically acceptable salt thereof: 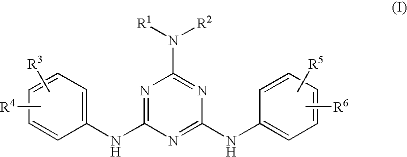
wherein the symbols are as follows;
R¹ and R², which may be the same or different, each represents H, OH, lower alkyl-O—, aryl-CO—, NH₂, lower alkyl-NH which may be substituted with OH, (lower alkyl)₂N, a lower alkyl which may be substituted, or a heterocyclic group which may be substituted; and
R³, R⁴, R⁵ and R⁶, which may be the same or different, each represents (i) H, (ii) CN, (iii) NO₂, (iv) halogen, (v) lower alkyl which may be substituted with (1) CN, (2) halogen, or (3) OH, (vi) cycloalkyl, (vii) aryl which may be substituted with lower alkyl, (viii) a heterocyclic group which may be substituted with lower alkyl, (ix) R⁷R⁸N-(wherein R⁷ and R⁸ may be the same or different, and each represents (1) H, (2) lower alkyl which may be substituted with aryl, or (3) R⁹—O—CO— (wherein R⁹ represents (1) H, or (2) lower alkyl which may be substituted with aryl)), (x) R¹⁰-T¹- (wherein R¹⁰ represents (1) H, (2) lower alkyl which may be substituted with aryl, HO—C₁₋₁₀ alkylene-O— or OH, or (3) aryl; and T¹ represents O or S), or (xi) R¹¹-T²- (wherein R¹¹ represents (1) OH, (2) R⁷R⁸N—, (3) lower alkyl-O—, (4) lower alkyl, (5) aryl, or (6) a heterocyclic group; and T² represents CO or SO₂).

6. The BEC1 potassium channel inhibitor according to claim 5, wherein the formula (I) is a compound wherein
R¹ and R², which may be the same or different, each represents H or lower alkyl which may be substituted; and
R³, R⁴, R⁵ and R⁶, which may be the same or different, each represents (i) H, (ii) halogen, or (iii) R¹⁰-T¹- (wherein R¹⁰ represents lower alkyl; and T¹ represents O),
or a pharmaceutically acceptable salt thereof.

7. A method of preventing and/or treating schizophrenia, comprising administering an effective amount of a BEC1 potassium channel inhibitor or a pharmaceutically acceptable salt thereof in a patient in need of such disease.

8. The method according to claim 7, wherein the BEC1 potassium channel inhibitor is a compound of the following formula (I) or a pharmaceutically acceptable salt thereof: 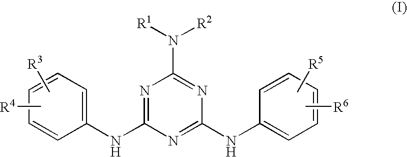
wherein the symbols are as follows;
R¹ and R², which may be the same or different, each represents H, OH, lower alkyl-O—, aryl-CO—, NH₂, lower alkyl-NH which may be substituted with OH, (lower alkyl)₂N, a lower alkyl which may be substituted, or a heterocyclic group which may be substituted; and
R³, R⁴, R⁵ and R⁶, which may be the same or different, each represents (i) H, (ii) CN, (iii) NO₂, (iv) halogen, (v) lower alkyl which may be substituted with (1) CN, (2) halogen, or (3) OH, (vi) cycloalkyl, (vii) aryl which may be substituted with lower alkyl, (viii) a heterocyclic group which may be substituted with lower alkyl, (ix) R⁷R⁸N-(wherein R⁷ and R⁸ may be the same or different, and each represents (1) H, (2) lower alkyl which may be substituted with aryl, or (3) R⁹—O—CO— (wherein R⁹ represents (1) H, or (2) lower alkyl which may be substituted with aryl)), (x) R¹⁰-T¹- (wherein R¹⁰ represents (1) H, (2) lower alkyl which may be substituted with aryl, HO—C₁₋₁₀ alkylene-O— or OH, or (3) aryl; and T¹ represents O or S), or (xi) R¹¹-T²- (wherein R¹¹ represents (1) OH, (2) R⁷R⁸N—, (3) lower alkyl-O—, (4) lower alkyl, (5) aryl, or (6) a heterocyclic group; and T² represents CO or SO₂).

9. The method according to claim 8, wherein the formula (I) is a compound wherein
R¹ and R², which may be the same or different, each represents H or lower alkyl which may be substituted; and
R³, R⁴, R⁵ and R⁶, which may be the same or different, each represents (i) H, (ii) halogen, or (iii) R¹⁰-T¹- (wherein R¹⁰ represents lower alkyl; and T¹ represents O),
or a pharmaceutically acceptable salt thereof.
